(19) United States
(12) Patent Application Publication
(10) Pub. No.: US 2026/0355537 A1
(43) Pub. Date: Nov. 12, 2026

(54) COSMETIC, AESTHETIC OR THERAPEUTIC METHODS OF ELECTROMAGNETIC RADIATION BASED DEVICES

(71) Applicant: AIWHITE LTD, Rehovot (IL)

(72) Inventors: Liraz SHLOMOFF, Rishon LeZion (IL); Tidhar TURGEMAN, Ramat Gan (IL); Tomer IZRAELI, Ra'anana (IL)

(21) Appl. No.: 19/725,254

(22) Filed: Jun. 30, 2026

Related U.S. Application Data
(63) Continuation of application No. PCT/IL2025/050031, filed on Jan. 9, 2025.
(60) Provisional application No. 63/619,371, filed on Jan. 10, 2024.

(51) Int. Cl. A61N 5/06; A61N 5/067

(52) U.S. Cl. CPC A61N 5/0625; A61N 5/067; A61N 2005/0606; A61N 2005/0627; A61N 2005/0659; A61N 2005/0661; A61N 2005/0663

(57) ABSTRACT

A responsive surface for use in an electromagnetic radiation (EMR)-based therapy, the responsive surface having a top face and a bottom face, wherein the bottom face is configured for intimate placement onto or adherence to a body region of a subject in a path of an EMR beam used in the EMR treatment of said body region, the responsive surface comprising a thermo-responsive material (or a photochromic material) that is unresponsive to the EMR and is capable of changing at least one visual parameter thereof in response to a change in the body region temperature.

### CROSS REFERENCE TO RELATED APPLICATIONS

This application is a Bypass Continuation of PCT Patent Application No. PCT/IL 2025/050031 having International filing date of Jan. 9, 2025, which claims the benefit of priority of U.S. Provisional Patent Application No. 63/619,371, filed Jan. 10, 2024, the contents of which are all incorporated herein by reference in their entirety.

### TECHNOLOGICAL FIELD

The invention generally contemplates improvements in treatments using devices based on electromagnetic radiation.

### BACKGROUND

Adapting exposure profile (time, intensity, wavelength, etc) of electromagnetic radiation-based devices, e.g., lasers, phototherapy, IPL, ultrasound, RF, Plasma devices etc., to achieve a suitable skin treatment, without causing skin damage, is mandatory for an efficient skin treatment, both from a point of view of treatment efficacy and patience safety. Part of the difficulty in selecting a proper operation protocol resides in the fact that electromagnetic radiation, are generally untraceable, causing inconsistency of treatment and at times lack of ability to properly irradiate all skin regions without causing damage due to re-exposure or overexposure. The associated deficiencies not only compromise the efficacy of the treatment and raise safety concerns, but they also come at the cost of patient generic treatment that lacks attention to a patient specific skin.

The discrepancy between the intended treatment plan and the actual therapeutic outcomes stems from a variety of factors. For example:
1. Uncontrolled and uneven radiation distances: This results in fluctuations in the intensity of radiation reaching the patient's skin, contributing to treatment inconsistencies.
2. Repetitive irradiation of the same treatment area: The lack of precise control may lead to unintended multiple exposures, potentially causing unintended effects or diminishing the overall effectiveness of the treatment.
3. Insufficient screening in non-targeted areas: The absence of proper screening measures may inadvertently expose areas not designated for treatment, posing risks to patient safety and potentially leading to undesirable side effects.
4. Inadequate adjustment of the treatment protocol based on skin tone: Failure to customize the treatment approach according to the patient's skin tone can result in suboptimal therapeutic outcomes.

To address these challenges and enhance the overall precision and effectiveness of electromagnetic radiation-based treatments, there is a pressing need for a product that provides a reliable means of gauging and controlling a radiation dosage applied to a skin region.

### SUMMARY OF THE INVENTION

The present invention pertains to a responsive surface or device designed for cosmetic, aesthetic, or therapeutic treatments involving body region exposure to electromagnetic radiation (EMR). The responsive surface or device may be configured as a sticker, a patch, or a dental splint that can respond to heat emitted from the body region, generating a visual indication of the treated area. Visual indication can facilitate customization of the treatment protocol to a subject's skin or teeth tone or region, optimizing the safety and/or cosmetic or therapeutic result. The responsive surface may minimize repetitive exposure of skin or teeth regions and ensure adequate exposure of desired areas, providing an exposure profile adjusted to a person's skin or teeth condition. The visual response may include changes in color, or intensity thereof using materials like pigments, dyes, or thermochromic substances. The product may be applied directly to the skin or teeth or to a substrate, and it can be disposable or reusable. The multilayered responsive surface or device may allow practitioners to administer EMR irradiation treatments with greater accuracy, ensuring both efficacy and safety.

Thus, an aspect of the invention pertains to a responsive surface for use in an electromagnetic radiation (EMR)-based therapy, the responsive surface having at least one face, wherein the face is configured for intimate placement onto or adherence to a body region a subject in a path of an EMR beam used in the EMR treatment of said body region, said responsive surface comprising a thermo-responsive material (or a photochromic material) that is unresponsive to the EMR and is capable of changing at least one visual parameter thereof in response to a change in the body region temperature.

Another aspect of the invention pertains to a responsive surface for use in an electromagnetic radiation (EMR)-based therapy, the responsive surface having a top face and a bottom face, wherein the bottom face is configured for intimate placement onto or adherence to a skin region of a subject in a path of an EMR beam used in the EMR treatment of the skin region, the responsive surface comprising a thermo-responsive material (or a photochromic material) that is unresponsive to the EMR and is capable of changing at least one visual parameter thereof in response to a change in the skin region temperature.

Another aspect of the invention pertains to a responsive surface for use in an EMR treatment, the responsive surface having a top face and a bottom face, wherein the bottom face is configured for intimate placement onto or adherence to a skin region of a subject and in a path of an EMR used in the treatment of the skin region, the responsive surface comprising a thermo-responsive material (or a photochromic material) having a first color indicative of a first temperature of the skin region and a second color indictive of a second temperature of the skin region, wherein the second temperature of the skin region is higher than the first temperature of the skin region and is lower than a predetermined temperature threshold indicative of excessive exposure to the EMR or indicative of an exposure level required for achieving treatment.

Another aspect of the invention pertains to a multilayered device comprising a responsive surface for use in an electromagnetic radiation (EMR)-based therapy, the responsive surface having a top face and a bottom face, wherein the bottom face is configured for intimate placement onto or adherence to a skin region of a subject in a path of an EMR beam used in the EMR treatment of the skin region, the responsive surface comprising a thermo-responsive material (or a photochromic material) that is unresponsive to the EMR and is capable of changing at least one visual parameter thereof in response to a change in the skin region temperature.

In one or more embodiments, the EMR-based therapy is selected from the group consisting of hair removal, teeth whitening, cellulitis, dry eye, elimination of small blood vessels, psoriasis, skin redness, scars, keloids, and more.

In one or more embodiments, the EMR-based therapy is teeth whitening.

In one or more embodiments, the EMR-based therapy is hair removal.

In one or more embodiments, the responsive surface is being in a form of a film or a flat pouch or a sticker associated with or comprising the at least one thermo-responsive or photochromic material.

In one or more embodiments, the body region is teeth and the responsive surface is being in a form of a dental splint.

In one or more embodiments, the bottom face is provided with an adhesive or means for holding the responsive surface in close proximity with the skin region such that a temperature change in the skin region due to EMR exposure induces a change in the color of the thermo-responsive material.

In one or more embodiments, the EMR treatment is a cometic, aesthetic or medical treatment that comprising delivery of EMR at an exposure or irradiance level that is at or above an exposure or irradiance level promoting one or more desired cosmetic, aesthetic or therapeutic effects to the skin.

In one or more embodiments, the thermo-responsive material is a material or a combination of materials that change color in response to a predetermined temperature threshold.

In one or more embodiments, a color change from a first color to a second color is indicative of reaching the temperature threshold above which the skin region is excessively exposed to EMR, or above which proper treatment may be achieved.

In one or more embodiments, the temperature-responsive material is selected amongst photoactive, photochromic and irreversible photochromic materials.

In one or more embodiments, the thermo-responsive material is selected from a triarylmethane, a diarylethene, an azastilbene, a nitrone, a quinone, a fulgide, a spiropyran, a naphthopyran, a spiro-oxazine, or a combination thereof.

In one or more embodiments, the diarylethene is stilbene.

In one or more embodiments, the EMR has a wavelength of between about 700 nm and about 1500 nm.

In one or more embodiments, the EMR is selected from an ultraviolet (UV), an infrared (IR), or a laser.

In one or more embodiments, the bottom face comprises an adhesive and the top face comprises the thermo-responsive material (or a photochromic material).

In one or more embodiments, the responsive surface further comprising barrier surfaces comprising a blocking material that accommodates the thermo-responsive material (or a photochromic material) therein.

In one or more embodiments, the responsive surface is configured to minimize or diminish repetitive exposure of skin regions. In one or more embodiments, the responsive surface is configured to ensure adequate EMR exposure of a desired skin region. In one or more embodiments, the responsive surface is configured to provide an exposure or irradiation profile adjusted to a person's skin tone.

In one or more embodiments, the visual response is used to monitor the progress of the treatment. In one or more embodiments, the visual response is used to determine an endpoint of the treatment. In one or more embodiments, the responsive surface comprises one or more layers. In one or more embodiments, the responsive surface is configured to be applied directly to the skin area to be treated. In one or more embodiments, the responsive surface is configured to be applied to a substrate that is then applied to the skin area to be treated. In one or more embodiments, the responsive surface is configured to be removed from the skin area after the treatment.

In one or more embodiments, the responsive surface is configured to be disposable after a single use.

In one or more embodiments, the responsive surface is configured to be reusable for multiple treatments.

In yet another aspect, the invention pertains to a process for laser treatment or phototherapy of a subject, the process comprising exposing a body region provided with a responsive surface with an EMR selected to cause at least one cosmetic or therapeutic improvement of the body region, wherein the responsive surface is configured to change color upon increase in the body region temperature that is greater than determined for the patient, whereby upon color change, exposure to EMR at the body region is arrested.

In yet another aspect, the invention pertains to a process for laser treatment or phototherapy of a subject, the process comprising exposing a skin region provided with a responsive surface with an EMR selected to cause at least one cosmetic or therapeutic improvement of the skin region, wherein the responsive surface is configured to change color upon increase in the skin region temperature that is greater than determined for the patient, whereby upon color change, exposure to EMR at the skin region is arrested.

In one or more embodiments, the body region is a skin region or at least one tooth.

In one or more embodiments, the skin region is a skin region to be treated.

In one or more embodiments, the skin region is a region of the skin next or in a vicinity of the region to be treated and which is used for determining the actual exposure parameters to be used.

In one or more embodiments, the process comprises providing a responsive surface as herein disclosed.

In one or more embodiments, the responsive surface is provided in a form of a sticker. In one or more embodiments, the responsive surface is provided in a form of a dental splint.

In yet another aspect, the invention provides a process for determining at least one patient specific parameter associated with an EMR exposure of a body region of the patient undergoing cosmetic or therapeutic EMR treatment, the process comprising exposing or irradiating a body region provided with a responsive surface with an EMR selected to cause at least one cosmetic or therapeutic improvement of the body region, and changing at least one parameter associated with the EMR until the responsive surface undergoes a color change, wherein the color change defines a temperature threshold indicative of an excessive exposure or indicative of an exposure level required for achieving treatment.

In one or more embodiments, the body region is a skin region or at least one tooth.

In one or more embodiments, the at least one parameter associated with the EMR is any one condition or operating parameter that affords the spatial and temporal control to the application.

In one or more embodiments, the at least one parameter is one or more of wavelength, light intensity, duration of exposure, and beam diameter of the incident light beam.

In yet another aspect, the invention pertains to a method of administering EMR irradiation treatment to a subject in need thereof comprising providing the herein responsive surface; applying the responsive surface to a skin area to be treated; exposing the skin area to EMR; monitoring the visual response of the responsive surface; and adjusting the treatment protocol based on the visual response.

In one or more embodiments, adjusting the treatment protocol comprises one or more of changing the EMR intensity, changing the EMR wavelength, changing the exposure time, changing the treatment area, or ceasing the treatment.

In one or more embodiments, the EMR-based therapy is selected from the group consisting of hair removal, teeth whitening, cellulitis, dry eye, elimination of small blood vessels, psoriasis, skin redness, scars, keloids, and more.

In one or more embodiments, the EMR-based therapy is teeth whitening.

In one or more embodiments, the EMR-based therapy is hair removal.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention may be more clearly understood upon reading of the following detailed description of non-limiting exemplary embodiments thereof, with reference to the following drawings, in which:

FIGS. 1A-B are schematic presentation of exemplary sticker devices comprising responsive surfaces for use in an electromagnetic radiation (EMR) skin therapy, according to some embodiments of the invention.

FIG. 2 illustrates the change in color over time of a responsive surface exposed to UV radiation, according to some embodiments of the invention.

FIGS. 3A-F are exemplary sticker devices comprising responsive surfaces and changes in color thereof in response to irradiation at 1064 nm, according to some embodiments of the invention.

FIGS. 4A-B illustrate an exemplary splint device comprising a responsive surface for use in an electromagnetic radiation (EMR) dental therapy, according to some embodiments of the invention.

### DETAILED DESCRIPTION

The inventors of the technology disclosed herein have developed and now disclose a product for use in method of cosmetic, aesthetic or therapeutic treatment that involves a body region (e.g., a skin region or teeth) exposure to electromagnetic radiation (EMR) including for example laser, ultraviolet (UV), infrared (IR), radiofrequency (RF), plasma, ultrasound or any other source of radiation acceptable in the field. The product of the invention is structured and configured for minimizing or diminishing repetitive exposure of skin or teeth regions, ensures adequate exposure of the desired skin or teeth regions, with an exposure or an irradiation profile that is fully adjusted to the person skin or teeth tone. The product of the invention thus enables practitioners to administer EMR irradiation treatments with greater accuracy, ensuring both efficacy and safety of treatment.

More specifically, the innovative product developed to address the challenges in EMR treatment is a cutting-edge smart formulation which may be in a form of a sticker or a responsive surface designed for application on the subject's skin. The innovative product may be in a form of a dental splint designed for application on the subject's teeth. This product serves as a crucial intermediary, effectively creating a buffer between an incident EMR, such as a light beam (IR/UV), tailored to achieve treatment and the skin or teeth area to be treated. It stands out by intelligently responding to heat emitted from the subject's skin or teeth, generating a visual indication of the treated area at the precise radiation level required as per the treatment protocol. Furthermore, the product introduces a visual response which may be used to facilitate the customization of the treatment protocol to the subject skin or teeth tone or skin or teeth region, thereby optimizing the cosmetic or therapeutic result.

In a first of its objects, the invention provides a responsive surface for use in EMR treatment, the responsive surface having a top face and a bottom face, wherein the bottom face is configured for intimate placement onto or adherence to a skin region of a subject in a path of an EMR, e.g., light beam used in the treatment of said skin region, said responsive surface comprising a thermo-responsive material (or a photochromic material) that is unresponsive to the EMR and is capable of changing at least one visual parameter thereof in response to a change in the skin region temperature.

The invention further provides a responsive surface for use in an EMR-based treatment, the responsive surface having a top face and a bottom face, wherein the bottom face is configured for intimate placement onto or adherence to a skin region of a subject and in a path of an EMR, e.g., light beam used in the treatment of said skin region, said responsive surface comprising a thermo-responsive material (or a photochromic material) having a first color indicative of a first temperature of the skin region and a second color indictive of a second temperature of the skin region, wherein the second temperature of the skin region is higher than the first temperature of the skin region and is lower than a predetermined temperature threshold indicative of excessive exposure or radiation or indicative of an exposure or a radiation level required for achieving treatment.

In a first of its objects, the invention provides a responsive surface for use in EMR treatment, the responsive surface having a top face and a bottom face, wherein the bottom face is configured for intimate placement onto or adherence to a teeth region of a subject in a path of an EMR, e.g., light beam used in the treatment of said teeth region, said responsive surface comprising a thermo-responsive material (or a photochromic material) that is unresponsive to the EMR and is capable of changing at least one visual parameter thereof in response to a change in the teeth region temperature.

The invention further provides a responsive surface for use in an EMR-based treatment, the responsive surface having a top face and a bottom face, wherein the bottom face is configured for intimate placement onto or adherence to a teeth region of a subject and in a path of an EMR, e.g., light beam used in the treatment of said teeth region, said responsive surface comprising a thermo-responsive material (or a photochromic material) having a first color indicative of a first temperature of the teeth region and a second color indictive of a second temperature of the teeth region, wherein the second temperature of the teeth region is higher than the first temperature of the teeth region and is lower than a predetermined temperature threshold indicative of excessive exposure or radiation or indicative of an exposure or a radiation level required for achieving treatment.

As disclosed herein, the responsive surface is typically a film or a flat pouch which is associated with or comprises the at least one thermo-responsive or photochromic material, as defined. The responsive surface has a bottom face that may be provided with an adhesive or means for holding the surface in close proximity with a body region such that a temperature change in the body region due to EMR exposure, e.g., light irradiation, will cause a change in the color of the thermo-responsive material. The dimensions of the responsive surface may vary based, inter alia, on the body region to be covered, the amount of thermo-responsive material used, the means by which the material is contained in or associated to the responsive surface, etc.

The responsive surface may be provided as a thin film of a material (such as a polymeric material) that is embedded or associated with the thermo-responsive or photochromic material. Alternatively, it may be provided in a form of a pouch in which the thermo-responsive or photochromic material may be distributed or contained. Irrespective of the way by which the thermo-responsive or photochromic material is provided, the responsive surface may be partially or fully transparent or may be colored white for ease of visualization of the temperature-inducted color change.

In some configurations, the thermo-responsive or photochromic material may be provided as a coating layer on top of an adhesive surface to provide an adhesive or a sticker film that can be securely placed on the skin or teeth region. In some cases, the thermo-responsive or photochromic material may be provided embedded in a polymeric film or a sticker.

The responsive surface may be a single layer or single film device of a multilayered device whereby different materials occupy different layers thereof. For example, one layer may be a layer of a first thermo-responsive or photochromic material and another layer may be of a second thermo-responsive or photochromic material. Other multilayered devices may comprise a layer selected to increase or minimize heat dissipation or heat conduction, and another may be a layer of a conductive material.

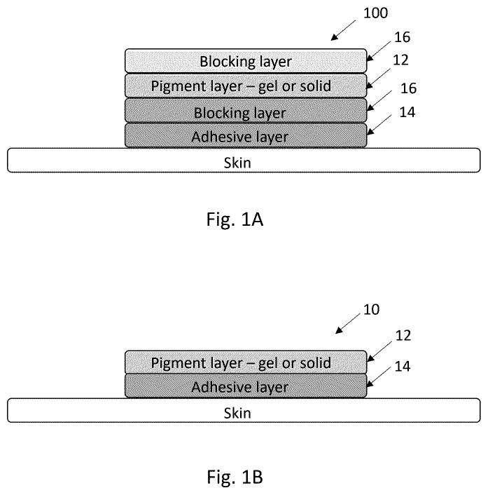

Exemplary cross section of layered devices 10 and 100 comprising responsive surfaces are depicted in FIGS. 1A-B. In the exemplary responsive surface 12, the pigment layer comprising the temperature-responsive material may be provided between two sheets or films of blocking materials or laminating materials 16 which may comprise functional materials, as shown in FIG. 1A. The bottom face 14 of the responsive surface 12 may be provided with an adhesive layer for placing onto a skin region of the subject.

There may be two or more blocking layers 16 which may be similar or may be different comprising different structure and/or materials. In an exemplary device 10 the responsive surface 12 may be solid, eliminating the need for a blocking layer 16 (FIG. 1B). In exemplary devices 100, where the responsive surface 12 is a semi-solid composition (e.g., a gel), the blocking layers 16 may function as barriers for accommodating therein the responsive surface 12.

In some configurations, the responsive surface may be adapted or configured as a sensor element for passively or actively reporting a change in its temperature or conductance to a control unit operable to carry out the EMR exposure or irradiation protocol. In an example, the responsive layer may be configured to be read or analyzed by an electronic, optical or a photoelectric device to determine a change in its color, conductance or any other measurable parameter which may be selected to indicate a temperature change.

The versatility of the responsive surface shines through in its ability to optimize a wide range of EMR-based treatment, e.g., laser treatments or phototherapy treatment protocols, including but not limited to hair removal, teeth whitening, cellulitis, dry eye, elimination of small blood vessels, psoriasis, skin redness, scars, keloids, and more. Moreover, the responsive surface, being in a form of e.g., a sticker/patch, can be applied to internal organs during surgeries, including laparoscopic, arthroscopic, or open procedures that involve various energy devices. By offering a solution that enhances accuracy and customization, the responsive surface is poised to revolutionize current practices in diverse medical and cosmetic applications.

The responsive surface is generally configured and adapted for any cosmetic or therapeutic method involving photoirradiation. As used herein, the term “electromagnetic radiation, EMR, treatment” encompasses any cometic, aesthetic or medical treatment that involves delivery of EMR, such as light energy at an exposure or irradiance level that is at or above an exposure or irradiance level that can promote one or more desired cosmetic, aesthetic or therapeutic effects to the body region. The exposure or irradiance level may or may not be below a level that can cut, cauterize, and/or ablate a biological tissue and may depend on the desired end result. Methods involving delivery of light energy may be tailored for, e.g., treatment of psoriasis, treatment of eczema, treatment of itch, treatment of vitiligo, treatment of morphea, removal of scars, removal of tattoos, hair removal and others.

In view of the broad use of EMR-based treatments, mainly involving laser treatments or phototherapy, the responsive surface may be tailored as a generic device suitable for use on any body region of a subject body and with any type of radiation used, or may be tailored for specific use or a specific body region. In a typical method of use, a responsive surface is positioned on a body region of the subject and the body region is exposed to EMR or irradiated with a light beam at an exposure or irradiance level that are predetermined. The thermo-responsive or photochromic material may be thus preselected to be unreactive or unresponsive under the exposure or irradiation conditions, namely the particular EM source used, the exposure level, the exposure time, etc, but undergo a color change due to an increase in the temperature of the body region during the exposure or irradiation period. The responsive surface incorporates a sophisticated chemical sensor that is designed to detect heat emitted from the subject skin or teeth during irradiation and as a result change color, but will not trigger a color change when the temperature of its top face changes for any reason.

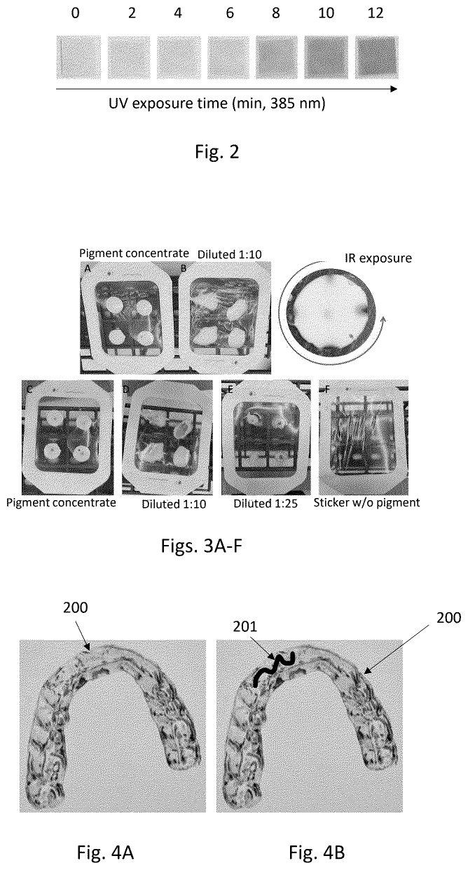

The thermo-responsive material is generally a material or a combination of materials that changes color when heated above a certain predetermined temperature. The threshold temperature may be reached immediately or after a certain time period, as demonstrated in FIG. 2 for a responsive surface exposed to UV radiation over time.

The heating or temperature increase is typically a temperature increase from a skin temperature to a skin temperature that is higher and a result of overheating of the skin due to the EMR exposure or light irradiation. The heating or temperature increase is typically a temperature increase from a teeth temperature to a teeth temperature that is higher and a result of overheating of the teeth due to the EMR exposure or light irradiation. As disclosed herein, the change in color is typically visible, but in some cases may be detected by proper sensing means.

The color change is typically from a first color that defines or is indicative of a first temperature of the body region prior to irradiation, and which is safe or which is not intended to induce any cosmetic or therapeutic change. The transition from the first color to the second color indicative of a second temperature of the skin or teeth region that is higher may be gradual or occur at once. The color change from the first color to the second color may be indicative of reaching a threshold temperature above which the skin or teeth region will undergo excessive radiation. Alternatively, the color change may be indicative of a radiation level required for achieving treatment. In other words, the color change may be indicative of the proper EMR conditions necessary to achieve an effective and safe treatment.

The change due to an increase in the skin temperature is demonstrated in the sticker devices shown in FIGS. 3A-F. Photos A and B show stickers according to the invention not placed on a skin-mimicking surface. A color change is not visible following irradiation at 1064 nm due to the fact that the surface did not change its temperature during the irradiation period. Photo F showed no color change as the sticker contained no temperature-responsive material. Photos C through E demonstrate a visible color change even where the amount of the temperature-responsive material was diluted.

FIGS. 4A-B illustrate yet another exemplary responsive surface in the form of a dental splint 200. The pigment layer comprising the temperature-responsive material may be provided as a single layer from which the dental splint is produced or between additional layers. For example, the dental splint 200 may be manufactured from a polymeric material mixed with the temperature-responsive material, providing a single-layer product. The dental splint 200 is positioned over the teeth prior to EMR treatment. When, due to an EMR beam that hits a certain region or spot on a tooth, temperature increases above a certain threshold, color change 201 will appear as illustrated in FIG. 4B, indicating that irradiation at that tooth region should be arrested.

It should be emphasized that the responsive surface is configured to undergo a color change only due to a temperature change in the skin region provided or coated with the responsive surface. A temperature change at a top face of the responsive surface, or EMR or light irradiation by a light beam will not induce a color change. In other words, the thermo-responsive material is unresponsive to the EMR, e.g., a light beam and is capable of changing at least one visual parameter thereof only in response to a change in the skin region temperature. Thermo-or temperature-responsive materials capable of changing color upon an increase in the temperature may be selected amongst photoactive, photochromic and irreversible photochromic materials, as known in the art. As used herein the term “hotoactive material” pertains to a substance that undergoes a physical or chemical change when exposed to light, typically ultraviolet (UV), visible, or infrared radiation. Photoactive materials can absorb photons and use the energy to drive various processes, such as electronic excitation, chemical reactions, or structural changes. In an embodiment of the invention, the photoactive material is a photochromic material, i.e., a substance that changes color or opacity when exposed to light. In an embodiment of the invention, the photoactive material is an irreversible photochromic material, i.e., a material that undergoes a permanent change in color or optical properties upon exposure to light. In an embodiment of the invention, the photoactive material is a reversible photochromic material. Unlike reversible photochromic materials, which can switch back to their original state when the light source is removed or under different environmental conditions, irreversible photochromic materials do not revert to their initial state after the light exposure ceases.

Specific materials may be selected amongst triarylmethanes; diarylethenes, e.g., stilbenes; azastilbenes; nitrones; quinones; fulgides; spiropyrans; naphthopyrans; spiro-oxazines; and others.

The temperature difference between the first body region temperature and the second body region temperature may differ and is generally such that the second temperature is not destructive or injurious to the body region. In some cases, and depending, inter alia, on the subject skin tone, the subject ability to sustain pain, the effective temperature for achieving the cosmetic or therapeutic result, the wavelength used, etc, the second temperature may be set to be below 45 °C. The temperature difference between first skin temperature, being typically between 33 °C and 37 °C, and the second skin temperature, being typically below 45 °C, may be between 10 and 12 °C In some cases, the temperature change may be even smaller. For example, the temperature difference may be between 3 °C and 12 °C, between 5 °C and 12 °C, between 7 °C and 12 °C, or between 8 °C and 12 °C. Each possibility represents a separate embodiment of the invention. Thus, depending for example on the first temperature and the temperature increase to be monitored, defining a temperature difference, the selection of a proper temperature-responsive material may be made.

The invention further provides a process for EMR treatment, e.g., laser treatments or phototherapy, of a patient, the process comprising exposing or irradiating a skin region provided with a responsive surface with an EMR such as a light beam selected to cause at least one cosmetic or therapeutic improvement of the skin region, wherein the responsive surface is configured to change color upon increase in the skin region temperature that is greater than determined for the patient, whereby upon color change, irradiation at the skin region is arrested.

In some embodiments, the skin region is a skin region to be treated and in other cases the skin region is a region of the skin next or in a vicinity of the region to be treated and which is used for determining the actual exposure or irradiation parameters to be used.

The invention further provides a process for EMR treatment, e.g., laser treatments or phototherapy, of a patient, the process comprising exposing or irradiating a teeth region provided with a responsive surface with an EMR such as a light beam selected to cause at least one cosmetic or therapeutic improvement of the teeth region, wherein the responsive surface is configured to change color upon increase in the teeth region temperature that is greater than determined for the patient, whereby upon color change, irradiation at the teeth region is arrested.

In some embodiments, the teeth region is a teeth region to be treated and in other cases the teeth region is a region of the teeth next or in a vicinity of the region to be treated and which is used for determining the actual exposure or irradiation parameters to be used.

In some embodiments, the process comprises providing a responsive surface as disclosed herein. In some embodiments, the process comprises applying, attaching, associating or adhering said responsive surface to the skin region to be treated by EMR.

In some embodiments, the responsive surface is provided in a form of a sticker.

The invention further provides a process for determining at least one patient specific parameter associated with an EMR, e.g., light beam radiation, of a skin region of the patient undergoing cosmetic or therapeutic EMR treatment, e.g., laser treatments or phototherapy treatment, the process comprising exposing or irradiating a skin region provided with a responsive surface with an EMR such as a light beam selected to cause at least one cosmetic or therapeutic improvement of the skin region, and changing at least one parameter associated with the EMR exposure until the responsive surface undergoes a color change, wherein said color change defines a temperature threshold indicative of an excessive EMR exposure or indicative of an EMR exposure level required for achieving treatment.

In some embodiments, the process comprises providing a responsive surface as disclosed herein. In some embodiments, the process comprises applying, attaching, associating or adhering said responsive surface to the teeth region to be treated by EMR.

In some embodiments, the responsive surface is provided in a form of a dental splint.

The invention further provides a process for determining at least one patient specific parameter associated with an EMR, e.g., light beam radiation, of a teeth region of the patient undergoing cosmetic or therapeutic EMR treatment, e.g., laser treatments or phototherapy treatment, the process comprising exposing or irradiating a teeth region provided with a responsive surface with an EMR such as a light beam selected to cause at least one cosmetic or therapeutic improvement of the teeth region, and changing at least one parameter associated with the EMR exposure until the responsive surface undergoes a color change, wherein said color change defines a temperature threshold indicative of an excessive EMR exposure or indicative of an EMR exposure level required for achieving treatment.

In some embodiments, the EMR is light radiation in the UV or IR regimes, wherein the light may be selected based on its wavelength which can significantly affect the extent of its cosmetic or therapeutic end result. In some embodiments, light irradiation may be in the ultraviolet regime (100-400 nm) or the infrared regime (700-2500 nm). For example, the irradiation may have a wavelength of between 100-2500 nm. The irradiation may have a wavelength of between 100-300 nm, 300-500 nm, 400-500, 500-700 nm, 700-1200 nm, 700-1100 nm, or 700-1000 nm. Each possibility represents a separate embodiment of the invention.

In some embodiments, the at least one parameter associated with the EMR or light beam radiation is any one condition or operating parameter that affords the spatial and temporal control to the application. The at least one parameter may include one or more of wavelength, light intensity, duration of exposure, and beam diameter of the incident light beam.

Responsive surfaces of the invention may be regarded as once-usable or reusable devices depending, inter alia, on the type of treatment and the type of thermos-responsive material used. Irrespectively, devices of the invention may be used for a variety of purposes.

Quantitative Skin Tone Identification: The device incorporates a marker or chemical component designed to respond to the energy emitted from the patient's skin. The result is a precise and quantitative translation of the patient's skin tone, providing a comprehensive understanding that serves as a foundational element for customized treatment protocols.

Thermally Responsive Formulation/Patch: An integral component is the patch that is provided or equipped with the marker or chemical component responsive to specific temperatures emitted from the patient's skin. Tailored to therapeutic requirements, such as hair removal or scar treatment, the patch ensures enhanced treatment efficiency. Notably, it mitigates the risk of radiation overdose, reducing the occurrence of skin burns and minimizing the potential for increased cancer risks. This dual-functionality contributes to the overall safety and effectiveness of the treatment process.

Electronic Eye for Laser Treatments or Phototherapy Devices: the Device may integrate an advanced electronic eye with laser treatments or phototherapy devices. This electronic eye serves a dual purpose: firstly, it accurately detects the intensity and rate of change of the chemical marker, providing a quantitative assessment of the patient's skin tone. Additionally, it monitors changes in the marker throughout the treatment.

Collectively, the device of the invention not only advances the precision and safety of laser treatments or phototherapy treatments by quantifying skin tone and optimizing therapeutic responses but also lays the groundwork for advancements in automated treatment processes, offering a comprehensive and forward-looking approach to medical and cosmetic interventions. The uniqueness of the technology may be summarized as follows:

Precision in Laser Treatments or phototherapy Treatment Optimization: the device groundbreaking innovation lies in its ability to optimize laser treatments or phototherapy treatments by meticulously aligning the treatment protocol with the patient's unique skin tone. This is achieved through the application of visible markers that serve as a guide for the therapist or, in the future, the electronic eye of an advanced robotic arm. This tailored approach ensures a heightened level of precision in treatment delivery.

Mitigation of Treatment Risks: Addressing a critical concern in current laser treatments or phototherapy practices, the system significantly minimizes the risks associated with treatment. By introducing markers that respond to specific temperatures, it effectively prevents the potential dangers of radiation overdose and excessive tissue heating. The absence of visible cues for treated areas, common in IR/UV rays, is intelligently overcome, thereby mitigating the risk of skin irritation or burns. This innovation represents a substantial leap in ensuring the safety and well-being of patients undergoing laser treatments or phototherapy.

Distinctive Material Composition (combination of polymeric film+adhesive material+thermochromic mixture or other temperature sensitive molecule): The device uniqueness is further underscored by its distinctive material composition, featuring a combination of a sensor with either a polymer for stickers or a specialized formulation for direct application to the patient's skin. This unique pairing of sensor and material ensures a seamless and effective integration, facilitating the accurate detection of skin tone and temperature changes during treatment.

Treatment Protocol Standardization: Another key aspect of innovation lies in the standardization of treatment protocols. In the current landscape, variations in laser treatments or phototherapy devices, including different wavelengths, energy intensities, and pulse rates, can lead to significant differences in treatment outcomes. By incorporating sensors, the system introduces a groundbreaking standardization approach. Regardless of the device used, the sensor's color change serves as a universal indicator, signifying the attainment of the required energy for a specific treatment. This standardization ensures consistency and efficacy across diverse laser treatments or phototherapy devices, promoting a streamlined and reliable treatment experience for practitioners and patients alike.

As used herein the terms ‘a’ and ‘an’ may mean ‘one’ or ‘more than one’.

As used herein the terms ‘comprising’, ‘including’, ‘containing’, ‘featuring’, ‘having’ and any forms of the terms thereof are inclusive and open ended and do not exclude additional elements or method steps, which are not recited.

The term ‘consisting essentially of’ means that the scope is limited to the specified elements and those that do not materially affect the basic and novel characteristic(s) of the claimed device and materials.

Each of the phrases ‘consisting of’ and ‘consists of’, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, 'from 2 to 4′, ‘from 2 to 6’, ‘from 3 to 6’, etc., and individual numerical values, such as ‘1’, ‘1.3’, '2′, '2.8′, ‘3’, ‘3.5’, ‘4’, ‘4.6’, ‘5’, ‘5.2’, and ‘6’, within the stated or described numerical range of 'from 1 to 6′. This applies regardless of the numerical breadth, extent, or size, of the stated or described numerical range.

All ranges disclosed herein include the endpoints. The use of the term “or” shall be construed to mean “and/or” unless the specific context indicates otherwise.

The term ‘about’, in some embodiments, refers to ±30 % of the stated numerical value. In further embodiments, the term refers to ±20 % of the stated numerical value. In yet further embodiments, the term refers to ±10 % of the stated numerical value.

1-28. (canceled)

29. A responsive surface for use in an electromagnetic radiation (EMR)-based therapy, the responsive surface having at least one face, wherein the face is configured for intimate placement onto or adherence to a body region of a subject in a path of an EMR beam used in the EMR treatment of said body region, said responsive surface comprising a thermo-responsive material (or a photochromic material) that is unresponsive to the EMR and is capable of changing at least one visual parameter thereof in response to a change in the body region temperature.

30. The responsive surface according to claim 29, for use in an EMR skin treatment, the responsive surface having a top face and a bottom face, wherein the bottom face is configured for intimate placement onto or adherence to a skin region of a subject and in a path of an EMR used in the treatment of said skin region, said responsive surface comprising a thermo-responsive material (or a photochromic material) having a first color indicative of a first temperature of the skin region and a second color indictive of a second temperature of the skin region, wherein the second temperature of the skin region is higher than the first temperature of the skin region and is lower than a predetermined temperature threshold indicative of excessive exposure to the EMR or indicative of an exposure level required for achieving treatment.

31. The responsive surface according to claim 29, being in a form of a film or a flat pouch or a sticker associated with or comprising the at least one thermo-responsive or photochromic material.

32. The responsive surface according to claim 29, wherein the body region is teeth, and the responsive surface is being in a form of a dental splint.

33. The responsive surface according to claim 30, wherein the bottom face is provided with an adhesive or means for holding the responsive surface in close proximity with the skin region such that a temperature change in the skin region due to EMR exposure induces a change in the color of the thermo-responsive material, and wherein said top face comprises said thermo-responsive material (or a photochromic material).

34. The responsive surface according to claim 29, wherein the EMR treatment is a cometic, aesthetic or medical treatment that comprising delivery of EMR at an exposure or irradiance level that is at or above an exposure or irradiance level promoting one or more desired cosmetic, aesthetic or therapeutic effects to the body region.

35. The responsive surface according to claim 29, wherein the thermo-responsive material is a material or a combination of materials that change color in response to a predetermined temperature threshold.

36. The responsive surface according to claim 35, wherein a color change from a first color to a second color is indicative of reaching the temperature threshold above which the body region is excessively exposed to EMR, or above which proper treatment may be achieved.

37. The responsive surface according to claim 29, wherein the temperature-responsive material is selected amongst photoactive, photochromic and irreversible photochromic materials.

38. The responsive surface according to claim 29, wherein the thermo-responsive material is selected from a triarylmethane, a diarylethene, an azastilbene, a nitrone, a quinone, a fulgide, a spiropyran, a naphthopyran, a spiro-oxazine, or a combination thereof.

39. The responsive surface according to claim 38, wherein the diarylethene is stilbene.

40. The responsive surface according to claim 29, wherein the EMR has a wavelength of between about 700 nm and about 1500 nm.

41. The responsive surface according to claim 29, wherein the EMR is selected from an ultraviolet (UV), an infrared (IR), or a laser.

42. The responsive surface according to claim 33, further comprising barrier surfaces comprising a blocking material that accommodates the thermo-responsive material (or a photochromic material) therein.

43. A process for laser treatment or phototherapy of a subject, the process comprising exposing a body region provided with a responsive surface with an EMR selected to cause at least one cosmetic or therapeutic improvement of the body region, wherein the responsive surface is configured to change color upon increase in the body region temperature that is greater than determined for the patient, whereby upon color change, exposure to EMR at the body region is arrested.

44. The process according to claim 43, wherein the body region is a skin region or at least one tooth.

45. The process according to claim wherein the skin region is a skin region to be treated, or a region of the skin next or in a vicinity of the region to be treated and which is used for determining the actual exposure parameters to be used.

46. The process according to claim 44, wherein the at least one tooth is a teeth region to be treated.

47. The process according to claim 43, wherein the process comprises providing a responsive surface for use in an electromagnetic radiation (EMR)-based therapy, the responsive surface having at least one face, wherein the face is configured for intimate placement onto or adherence to a body region of a subject in a path of an EMR beam used in the EMR treatment of said body region, said responsive surface comprising a thermo-responsive material (or a photochromic material) that is unresponsive to the EMR and is capable of changing at least one visual parameter thereof in response to a change in the body region temperature.

48. A process for determining at least one patient specific parameter associated with an EMR exposure of a body region of the patient undergoing cosmetic or therapeutic EMR treatment, the process comprising exposing or irradiating a body region provided with a responsive surface with an EMR selected to cause at least one cosmetic or therapeutic improvement of the body region, and changing at least one parameter associated with the EMR until the responsive surface undergoes a color change, wherein said color change defines a temperature threshold indicative of an excessive exposure or indicative of an exposure level required for achieving treatment, wherein the at least one parameter associated with the EMR is any one condition or operating parameter that affords the spatial and temporal control to the application, and wherein the at least one parameter is one or more of wavelength, light intensity, duration of exposure, and beam diameter of the incident light beam.
